(19) United States
(12) Patent Application Publication
(10) Pub. No.: US 2019/0175035 A1
(43) Pub. Date: Jun. 13, 2019

(54) DEVICES AND METHODS FOR DETERMINING PULSE WAVE VELOCITY BASED ON CHANGES IN VESSEL DIAMETER

(71) Applicant: KONINKLIJKE PHILIPS N.V., Eindhoven (NL)

(72) Inventors: Arjen VAN DER HORST, Tilburg (NL); Charles Frederik SIO, Eindhoven (NL); Maarten Petrus Joseph KUENEN, Veldhoven (NL)

(73) Assignee: Koninklijke Philips N.V., Eindhoven (NL)

(21) Appl. No.: 16/301,016

(22) PCT Filed: May 19, 2017
(86) PCT No.: PCT/EP2017/062196

§ 371 Date: Nov. 13, 2018

(30) Foreign Application Priority Data
May 20, 2016 (EP) 16170662.7
Jun. 29, 2016 (EP) 16176911.2

(51) Int. Cl. A61B 5/024; A61B 8/12; A61B 8/02

(52) U.S. Cl. CPC A61B 5/02427; A61B 8/02; A61B 8/12

(57) ABSTRACT

Devices, systems and methods for pulse wave velocity determination in a renal artery are disclosed. An intravascular system may be included with two or more sensors disposed a certain distance apart on a flexible, elongate member. The sensors may be configured to measure changes in measurement value of the renal artery, such as the diameter of the renal artery and/or a distance between the sensors and the vessel walls, with pulse waves moving through the renal arter. The difference in the time at which the sensors measure these changes and the distance between the sensors may be used to calculate pulse wave velocity.

### TECHNICAL FIELD OF THE INVENTION

Embodiments of the present disclosure relate generally to the field of medical devices and, more particularly, to devices, systems, and methods for determining pulse wave velocity.

### BACKGROUND OF THE INVENTION

Hypertension and its associated conditions, chronic heart failure (CHF) and chronic renal failure (CRF), constitute a significant and growing global health concern. Current therapies for these conditions span the gamut covering non-pharmacological, pharmacological, surgical, and implanted device-based approaches. Despite the vast array of therapeutic options, the control of blood pressure and the efforts to prevent the progression of heart failure and chronic kidney disease remain unsatisfactory.

Blood pressure is controlled by a complex interaction of electrical, mechanical, and hormonal forces in the body. The main electrical component of blood pressure control is the sympathetic nervous system (SNS), a part of the body's autonomic nervous system, which operates without conscious control. The sympathetic nervous system connects the brain, the heart, the kidneys, and the peripheral blood vessels, each of which plays an important role in the regulation of the body's blood pressure. The brain plays primarily an electrical role, processing inputs and sending signals to the rest of the SNS. The heart plays a largely mechanical role, raising blood pressure by beating faster and harder, and lowering blood pressure by beating slower and less forcefully. The blood vessels also play a mechanical role, influencing blood pressure by either dilating (to lower blood pressure) or constricting (to raise blood pressure).

The importance of blood pressure in the kidneys is amplified because of the central electrical, mechanical, and hormonal role the kidneys play. For example, the kidneys affect blood pressure by signaling the need for increased or lowered pressure through the SNS (electrical), by filtering blood and controlling the amount of fluid in the body (mechanical), and by releasing key hormones that influence the activities of the heart and blood vessels to maintain cardiovascular homeostasis (hormonal). The kidneys send and receive electrical signals from the SNS and thereby affect the other organs related to blood pressure control. They receive SNS signals primarily from the brain, which partially control the mechanical and hormonal functions of the kidneys. At the same time, the kidneys also send signals to the rest of the SNS, which may boost the level of sympathetic activation of all the other organs in the system, effectively amplifying electrical signals in the system and the corresponding blood pressure effects. From the mechanical perspective, the kidneys are responsible for controlling the amount of water and sodium in the blood, directly affecting the amount of fluid within the circulatory system. If the kidneys allow the body to retain too much fluid, the added fluid volume raises blood pressure. Lastly, the kidneys produce blood pressure regulating hormones including renin, an enzyme that activates a cascade of events through the renin-angiotensin-aldosterone system (RAAS). This cascade, which includes vasoconstriction, elevated heart rate, and fluid retention, may be triggered by sympathetic stimulation. The RAAS operates normally in non-hypertensive patients but may become overactive among hypertensive patients. The kidney also produces cytokines and other neurohormones in response to elevated sympathetic activation that may be toxic to other tissues, particularly the blood vessels, heart, and kidney. As such, overactive sympathetic stimulation of the kidneys may be responsible for much of the organ damage caused by chronic high blood pressure.

Thus, overactive sympathetic stimulation of the kidneys plays a significant role in the progression of hypertension, CHF, CRF, and other cardio-renal diseases. Heart failure and hypertensive conditions often result in abnormally high sympathetic activation of the kidneys, creating a vicious cycle of cardiovascular injury. An increase in renal sympathetic nerve activity leads to the decreased removal of water and sodium from the body, as well as increased secretion of renin, which leads to vasoconstriction of blood vessels supplying the kidneys. Vasoconstriction of the renal vasculature causes decreased renal blood flow, which causes the kidneys to send afferent SNS signals to the brain, triggering peripheral vasoconstriction and increasing a patient's hypertension. Reduction of sympathetic renal nerve activity, e.g., via renal neuromodulation or denervation of the renal nerve plexus, may reverse these processes.

Efforts to control the consequences of renal sympathetic activity have included the administration of medications such as centrally acting sympatholytic drugs, angiotensin converting enzyme inhibitors and receptor blockers (intended to block the RAAS), diuretics (intended to counter the renal sympathetic mediated retention of sodium and water), and beta-blockers (intended to reduce renin release). The current pharmacological strategies have significant limitations, including limited efficacy, compliance issues, and side effects.

As noted, renal denervation is a treatment option for resistant hypertension. However, the efficacy of renal denervation may be very variable between patients. Recent studies indicate that the velocity of the pressure/flow pulse (pulse wave velocity or PWV) inside the main renal artery may be indicative of the outcome of renal denervation. The PWV in patients with resistant hypertension may be very high (e.g., more than 20 m/s), which may make it difficult to determine the PWV in the relatively short renal arteries (e.g., 5-8 cm in length).

While the existing treatments have been generally adequate for their intended purposes, they have not been entirely satisfactory in all respects. The devices, systems, and associated methods of the present disclosure overcome one or more of the shortcomings of the prior art.

US 2010/0113949 A1 discloses systems and methods for the measurement of the velocity of a pulse wave propagating within a body lumen using an intravascular elongate medical device. The elongate medical device can include a data collection device configured to collect pulse wave data at a location within the lumen. The data collection device is communicatively coupled with a velocity measurement system and configured to output the collected data to the velocity measurement system. The velocity measurement system is configured to calculate the velocity of the pulse wave based on the collection data.

WO 99/34724 A2 relates to devices and methods for determining tubular wall properties for improved clinical diagnosis and treatment. Advantageously, tubular wall characteristics are recorded that correspond to the distensibility and compliance of the tubular walls. More specifically, the document provides for quantitative determination of the pressure wave velocity (PWV) of blood vessels, thereby characterizing, (inter alia), the Young modulus, the distensibility, the compliance, and the reflection coefficient of aneurysms, lesioned and non-lesioned parts of blood vessels.

P. Lurz et al., “Aortic pulse wave velocity as a marker for arterial stiffness predicts outcome of renal sympathetic denervation and remains unaffected by the intervention”, European Heart Journal, Vol. 36, No. Suppl. 1, Aug. 1, 2015, assess the impact of baseline arterial stiffness as assessed by aortic pulse wave velocity (PWV) on blood pressure (BP) changes after renal sympathetic denervation (RSD) for resistant arterial hypertension as well as the potential of RSD to at least partially reverse increased aortic stiffness.

### SUMMARY OF THE INVENTION

The present disclosure describes calculation of a physiological quantity known as a pulse wave velocity (PWV). The PWV represents the velocity of the pressure and flow waves that propagate through blood vessels of a patient as a result of the heart pumping. Recent studies indicate that the PWV within the renal artery, which is an artery that supplies blood to the kidney, is indicative of whether a therapeutic known as renal denervation will be successful in the patient. Renal denervation is used to treat hypertension. As described in more detail herein, PWV can be determined based on a diameter of the vessel. Also, PWV can be determined based on the distance from a sensor to the vessel walls and/or a change in the distance from a sensor to the vessel walls. Alternatively, PWV can be determined based on measurements of the diameter change perpendicular to the vessel axis, such as a velocity of the vessel walls. Two or more sensors can be attached a known distance apart to a flexible, elongate member that is positioned within the vessel. The sensors measure changes in the distance from the sensor to the vessel wall associated with blood pulses moving through the vessel. The difference in the time at which the sensors measure these changes and the distance between the sensors may be used to calculate pulse wave velocity. The calculated PWV for the patient can then be used to determine whether the patient is good candidate for treatment. For example, the PWV measurement result can be used to perform patient stratification for the renal denervation, before performing the treatment, by predicting the efficacy of renal denervation based on PWV.

In one embodiment, an apparatus for pulse wave velocity (PWV) determination in a vessel is provided. The apparatus includes an intravascular device configured to be positioned within the vessel, the intravascular device including a flexible elongate member having a proximal portion and a distal portion; a first imaging element coupled to the distal portion of the flexible elongate member; and a second imaging element coupled to the distal portion of the flexible elongate member at a position spaced from the first imaging element by a first distance along a length of the flexible elongate member. The first imaging element is configured to monitor a measurement value within the vessel, for instance, a distance from the first imaging element to the vessel walls (e.g., a diameter of the vessel) or a change in the distance from the first imaging element to the vessel walls (e.g., a change in diameter of the vessel), at a first location. The second imaging element is configured to monitor a measurement value within the vessel, for instance, a distance from the second imaging element to the vessel walls (e.g., a diameter of the vessel) or a change in the distance from the second imaging element to the vessel walls (e.g., a change in diameter of the vessel), at a second location spaced from the first location; and a processing system in communication with the intravascular device, the processing system configured to: receive a first data associated with the monitoring measurement value of the vessel at the first location within the vessel by the first imaging element; receive a second data associated with the monitoring of the measurement value of the vessel at the second location within the vessel by the second imaging element; and determine a pulse wave velocity of fluid within the vessel based on the received first and second data. The vessel is a renal artery and the sampling frequency of the first and the second imaging element is 10 kHz or higher, more preferably, 20 kHz or higher, most preferably, 40 kHz or higher.

Two or more imaging elements can be attached at a known distance apart to a flexible elongate member that is positioned within the vessel. The imaging elements measure distances to the vessel wall, at different times to determine, for example, at what times the distance to the wall vessel is at maximum. This difference in time of when the distance to the wall vessel is maximum for the two imaging elements and the distance between the imaging elements may be used to calculate pulse wave velocity.

In one embodiment, a method of determining pulse wave velocity (PWV) in a vessel is provided. The method includes monitoring a measurement value (e.g., a vessel diameter, a change in vessel diameter, a distance to a wall of the vessel, or a change in the distance to the wall of the vessel) at a first location of the vessel by a first imaging element; monitoring a measurement value (e.g., the vessel diameter, the change in the vessel diameter, the distance to the wall of the vessel, or the change in the distance to the wall of the vessel) at a second location of the vessel by a second imaging element, wherein the second location is spaced from the first location along a length of the vessel by a first distance; receiving a first data associated with the monitoring of the measurement value of the vessel at the first location by the first imaging element; receiving a second data associated with the monitoring of the measurement value of the vessel at the second location by the second imaging element; and determining a pulse wave velocity of fluid within the vessel based on the received first and second data. The vessel is a renal artery the sampling frequency of the first and the second imaging element is 10 kHz or higher, more preferably, 20 kHz or higher, most preferably, 40 kHz or higher.

An apparatus for pulse wave velocity (PWV) determination in a vessel is also provided. The apparatus includes at least one sensing element configured to: monitor a vessel wall at a first location of the vessel; and monitor a vessel wall at a second location of the vessel, wherein the second location is spaced from the first location along a length of the vessel by a first distance; a processing system in communication with the at least one imaging element, the processing system configured to: receive first data associated with the monitoring of the vessel wall at the first location; receive second data associated with the monitoring of the vessel wall at the second location; and determine a pulse wave velocity of fluid within the vessel based on the received first and second data.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagrammatic schematic view of an exemplary intravascular sensor system.

FIG. 2 is a diagrammatic schematic view of another exemplary intravascular sensor system.

FIG. 3 is a schematic diagram illustrating an intravascular device positioned within the renal anatomy.

FIG. 4 is a graph of pressure measurements associated with pulse waves travelling through a vessel.

FIG. 5A is a diagrammatic schematic view of an exemplary intravascular device within a vessel combined with a graph showing pressure curves within the vascular pathway.

FIG. 5B is a diagrammatic schematic view of the exemplary intravascular device of FIG. 5A combined with a graph showing pressure curves within the vessel at a second time.

FIG. 5C is a diagrammatic schematic view of the exemplary intravascular device of FIG. 5A combined with a graph showing pressure curves within the vessel at a third time.

FIG. 6 shows a comparison of two distance measurements associated with pulse waves travelling through a vessel at two different locations within the vessel.

FIG. 7A is a diagrammatic schematic view of an exemplary measurement device disposed outside a patient's body.

FIG. 7B is a diagrammatic schematic view of an exemplary measurement device disposed outside a patient's body.

FIG. 8 is a diagrammatic schematic view of an exemplary intravascular device within a branched vessel combined with a graph showing pressure curves within the vessel.

FIG. 9 is a flowchart illustrating a method of calculating a pulse wave velocity.

### DETAILED DESCRIPTION OF THE EMBODIMENTS

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The present disclosure relates generally to devices, systems, and methods for determining and measuring pulse wave velocity in a main renal artery prior to a renal denervation treatment. The velocity of the pressure/flow pulse (pulse wave velocity or PWV) inside the main renal artery may be predictive of the outcome of renal denervation. The PWV may be very high in resistant hypertension patients, which makes it very difficult to perform an accurate measurement of PWV in the relatively short renal arteries. Sensors positioned within a vessel may be used to determine the PWV in the vessel. However, the sampling frequency of the sensors may be a limiting factor when using this method in determining PWV in short vessels, such as the renal arteries. One method to determine the PWV is by utilizing the “water hammer” equation to calculate the PWV from simultaneous pressure and flow velocity measurements inside the vessel during a reflection free period (e.g., early systole):

$$PWV = \frac{1}{ρ} \frac{dP}{dU} \qquad ( 1 )$$

Or, alternatively, in case this reflection free period cannot be used the following relation may be used that determines the PWV by summation over the whole cardiac cycle:

$$PWV = \frac{1}{ρ} \sqrt{\frac{\sum {dP}^{2}}{\sum {dU}^{2}}} \qquad ( 2 )$$

with ρ being the blood density and P and U the pressure and velocity, respectively.

As noted, renal denervation is a treatment option for resistant hypertension. Recent studies indicate that the velocity of the pressure/flow pulse (pulse wave velocity or PWV) inside the main renal artery pre-treatment may be predictive of the outcome of renal denervation treatment. In some instances, embodiments of the present disclosure are configured to perform pulse wave velocity measurements of the renal artery for stratification of patients for renal artery denervation. Renal sympathetic activity may worsen symptoms of hypertension, heart failure, and/or chronic renal failure. In particular, hypertension has been linked to increased sympathetic nervous system activity stimulated through any of four mechanisms, namely (1) increased vascular resistance, (2) increased cardiac rate, stroke volume and output, (3) vascular muscle defects, and/or (4) sodium retention and renin release by the kidney. As to this fourth mechanism in particular, stimulation of the renal sympathetic nervous system may affect renal function and maintenance of homeostasis. For example, an increase in efferent renal sympathetic nerve activity may cause increased renal vascular resistance, renin release, and sodium retention, all of which exacerbate hypertension.

As an example, thermal neuromodulation by either intravascular heating or cooling may decrease renal sympathetic activity by disabling the efferent and/or afferent sympathetic nerve fibers that surround the renal arteries and innervate the kidneys through renal denervation, which involves selectively disabling renal nerves within the sympathetic nervous system (SNS) to create at least a partial conduction block within the SNS. Several forms of renal injury or stress may induce activation of the renal afferent signals (e.g., from the kidney to the brain or the other kidney). For example, renal ischemia, a reduction in stroke volume or renal blood flow, may trigger activation of renal afferent nerve activity. Increased renal afferent nerve activity results in increased systemic sympathetic activation and peripheral vasoconstriction (narrowing) of blood vessels. Increased vasoconstriction results in increased resistance of blood vessels, which results in hypertension. Increased renal efferent nerve activity (e.g., from the brain to the kidney) results in further increased afferent renal nerve activity and activation of the RAAS cascade, inducing increased secretion of renin, sodium retention, fluid retention, and reduced renal blood flow through vasoconstriction. The RAAS cascade also contributes to systemic vasoconstriction of blood vessel, thereby exacerbating hypertension. In addition, hypertension often leads to vasoconstriction and atherosclerotic narrowing of blood vessels supplying the kidneys, which causes renal hypoperfusion and triggers increased renal afferent nerve activity. In combination this cycle of factors results in fluid retention and increased workload on the heart, thus contributing to the further cardiovascular and cardio-renal deterioration of the patient.

Renal denervation, which affects both the electrical signals going into the kidneys (efferent sympathetic activity) and the electrical signals emanating from them (afferent sympathetic activity) may impact the mechanical and hormonal activities of the kidneys themselves, as well as the electrical activation of the rest of the SNS. Blocking efferent sympathetic activity to the kidney may alleviate hypertension and related cardiovascular diseases by reversing fluid and salt retention (augmenting natriuresis and diuresis), thereby lowering the fluid volume and mechanical load on the heart, and reducing inappropriate renin release, thereby halting the deleterious hormonal RAAS cascade before it starts.

By blocking afferent sympathetic activity from the kidney to the brain, renal denervation may lower the level of activation of the whole SNS. Thus, renal denervation may also decrease the electrical stimulation of other members of the sympathetic nervous system, such as the heart and blood vessels, thereby causing additional anti-hypertensive effects. In addition, blocking renal nerves may also have beneficial effects on organs damaged by chronic sympathetic over-activity, because it may lower the level of cytokines and hormones that may be harmful to the blood vessels, kidney, and heart.

Furthermore, because renal denervation reduces overactive SNS activity, it may be valuable in the treatment of several other medical conditions related to hypertension. These conditions, which are characterized by increased SNS activity, include left ventricular hypertrophy, chronic renal disease, chronic heart failure, insulin resistance (diabetes and metabolic syndrome), cardio-renal syndrome, osteoporosis, and sudden cardiac death. For example, other benefits of renal denervation may theoretically include: reduction of insulin resistance, reduction of central sleep apnea, improvements in perfusion to exercising muscle in heart failure, reduction of left ventricular hypertrophy, reduction of ventricular rates in patients with atrial fibrillation, abrogation of lethal arrhythmias, and slowing of the deterioration of renal function in chronic kidney disease. Moreover, chronic elevation of renal sympathetic tone in various disease states that exist with or without hypertension may play a role in the development of overt renal failure and end-stage renal disease. Because the reduction of afferent renal sympathetic signals contributes to the reduction of systemic sympathetic stimulation, renal denervation may also benefit other organs innervated by sympathetic nerves. Thus, renal denervation may also alleviate various medical conditions, even those not directly associated with hypertension.

The devices, systems, and methods described herein allow for the determination of PWV in the renal arteries. In particular, accurate determination of localized PWV values in the renal artery may be used to predict the effect of renal denervation in a patient and selection of patients for whom this procedure is likely beneficial.

The PWV may be predictive of the outcome of renal denervation in treating resistive hypertension. As described herein, the computing device can output the calculated PWV to a display. A clinician may make therapeutic and/or diagnostic decisions, taking the PWV into consideration, such as whether to recommend the patient for a renal denervation procedure. In some instances, the computer system can determine and output a therapy recommendation or a likelihood-of-success prediction to the display, based on the PWV and/or other patient data. That is, the computer system may utilize the PWV to identify which patients are more likely and/or less likely to benefit from renal denervation.

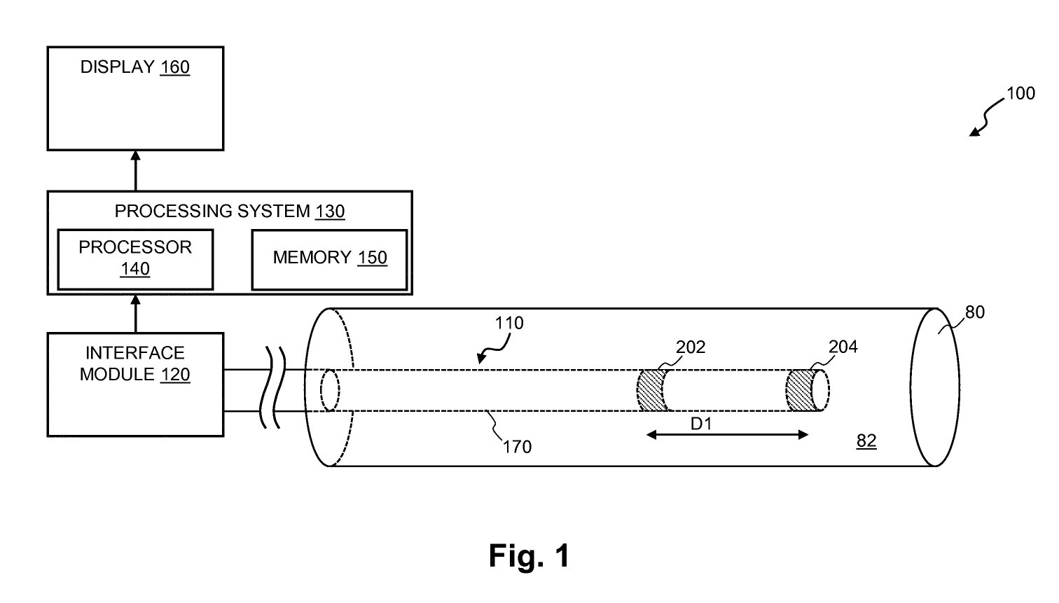

FIG. 1 is a diagrammatic schematic view of an exemplary intravascular system 100 according to some embodiments of the present disclosure. The intravascular system 100, which may be referred to as a stratification system, may be configured to perform pulse wave velocity (PWV) determination in a vessel 80 (e.g., artery, vein, etc.), for patient stratification for treatment purposes. For example, the PWV determination in the renal arteries may be utilized to determine whether a patient is suitable for renal artery denervation. The intravascular system 100 may include an intravascular device 110 that may be positioned within the vessel 80, an interface module 120, a processing system 130 having at least one processor 140 and at least one memory 150, and a display 160.

In some embodiments, the system 100 may be configured to perform pulse wave velocity (PWV) determination in a vessel 80 within a body portion. The intravascular system 100 may be referred to as a stratification system in that the PWV may be used for patient stratification for treatment purposes. For example, the PWV determination in the renal arteries may be utilized to determine whether a patient is suitable for renal artery denervation. Based on the PWV determination, the intravascular system 100 may be used to classify one or more patients into groups respectively associated with varying degrees of predicted therapeutic benefit of renal denervation. Any suitable number of groups or categories are contemplated. For example, the groups may include groups respectively for those patients with low, moderate, and/or high likelihood of therapeutic benefit from renal denervation, based on the PWV. Based on the stratification or classification, the system 100 can recommend the degree to which one or more patients are suitable candidates for renal denervation.

The vessel 80 may represent fluid-filled or surrounded structures, both natural and man-made. The vessel 80 may be within a body of a patient. The vessel 80 may be a blood vessel, as an artery or a vein of a patient's vascular system, including cardiac vasculature, peripheral vasculature, neural vasculature, renal vasculature, and/or or any other suitable lumen inside the body. For example, the intravascular device 110 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the heart, chambers or other parts of the heart, and/or other systems of the body. In addition to natural structures, the device intravascular 110 may be used to examine man-made structures such as, but without limitation, heart valves, stents, shunts, filters and other devices. Walls of the vessel 80 define a lumen 82 through which fluid flows within the vessel 80.

The vessel 80 may be located within a body portion. When the vessel 80 is the renal artery, the patient body portion may include the abdomen, lumbar region, and/or thoracic region. Generally, vessel 80 may be located within any portion of the patient body, including the head, neck, chest, abdomen, arms, groin, legs, etc.

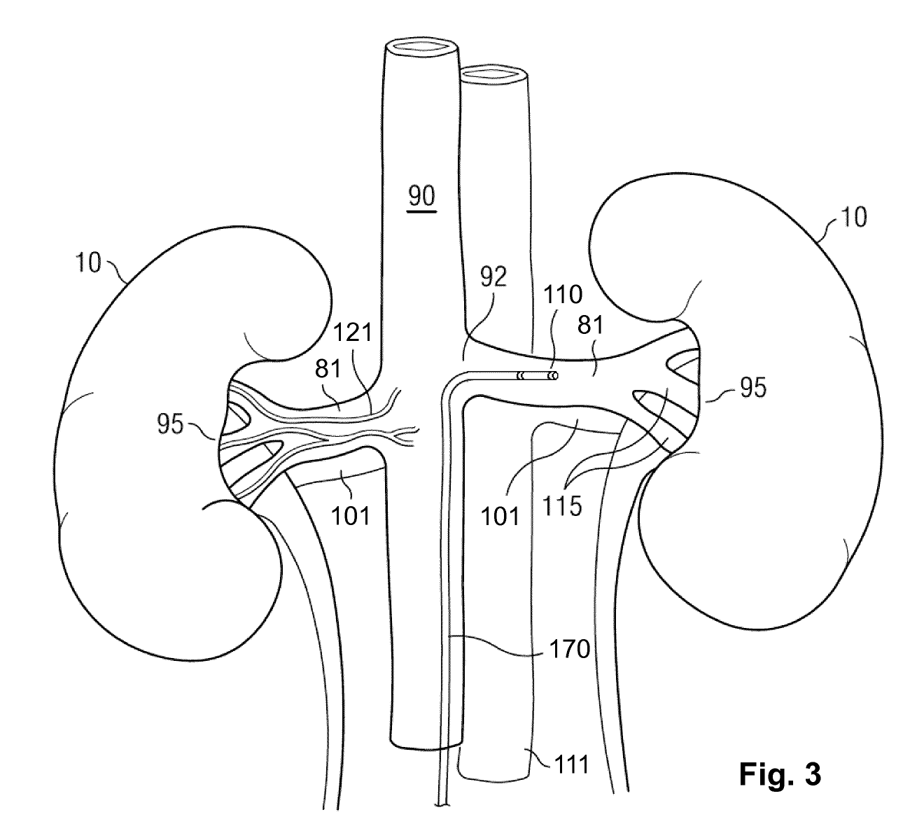

In some embodiments, the intravascular device 110 may include a flexible elongate member 170 such as a catheter, guide wire, or guide catheter, or other long, thin, long, flexible structure that may be inserted into a vessel 80 of a patient. In some embodiments, the vessel 80 is a renal artery 81 as shown in FIG. 3. While the illustrated embodiments of the intravascular device 110 of the present disclosure have a cylindrical profile with a circular cross-sectional profile that defines an outer diameter of the intravascular device 110, in other instances, all or a portion of the intravascular device may have other geometric cross-sectional profiles (e.g., oval, rectangular, square, elliptical, etc.) or non-geometric cross-sectional profiles. In some embodiments, the intravascular device 110 may or may not include a lumen extending along all or a portion of its length for receiving and/or guiding other instruments. If the intravascular device 110 includes a lumen, the lumen may be centered or offset with respect to the cross-sectional profile of the intravascular device 110.

The intravascular device 110, or the various components thereof, may be manufactured from a variety of materials, including, by way of non-limiting example, plastics, polytetrafluoroethylene (PTFE), polyether block amide (PEBAX), thermoplastic, polyimide, silicone, elastomer, metals, such as stainless steel, titanium, shape-memory alloys such as Nitinol, and/or other biologically compatible materials. In addition, the intravascular device may be manufactured in a variety of lengths, diameters, dimensions, and shapes, including a catheter, guide wire, etc. For example, in some embodiments the flexible elongate member 170 may be manufactured to have length ranging from approximately 115 cm-155 cm. In one particular embodiment, the flexible elongate member 170 may be manufactured to have length of approximately 135 cm. In some embodiments, the flexible elongate member 170 may be manufactured to have an outer transverse dimension or diameter ranging from about 0.35 mm-2.67 mm (1 Fr-8 Fr). In one embodiment, the flexible elongate member 170 may be manufactured to have a transverse dimension of 2 mm (6 Fr) or less, thereby permitting the intravascular device 110 to be configured for insertion into the renal vasculature of a patient. These examples are provided for illustrative purposes only, and are not intended to be limiting. Generally, the intravascular device 110 is sized and shaped such that it may be moved inside the vasculature (or other internal lumen(s)) of a patient such that the diameter and cross-sectional area of a vessel 80 may be monitored from within the vessel 80.

In some embodiments, the intravascular device 110 includes a sensor 202 and a sensor 204 disposed along the length of the flexible elongate member 170. The sensors 202, 204 may be configured to collect data about conditions within the vessel 80, and in particular, identify changes in the diameter of the vessel 80. In some embodiments, the sensors 202, 204 are ultrasound transducers, such as a CMUT, PMUT, PZT, single crystal ultrasound transducers, or other suitable ultrasound transducers. In this regard, the sensors 202, 204 may be part of a rotational intravascular ultrasound imaging arrangement or part of a phased array intravascular ultrasound arrangement.

As noted above, the imaging element can be a rotational intravascular ultrasound (IVUS) apparatus. More specifically, the sensors 202, 204 may be ultrasound transducers that rotate about the longitudinal axis of the intravascular device 110 with respect to the flexible elongate member 170. In this regard, a rotational drive cable or shaft may extend through the flexible elongate member 170 to the distal portion where the sensors 202, 204 are mounted.

In some embodiments, the sensors 202, 204 may be part of an array of ultrasound transducers (e.g., 32, 64, 128, or other number transducers) disposed on the flexible elongate member 170. This may allow for the generation of two or more imaging modes (such as an A-mode and a B-mode), which may allow for the measurement of propagating wall distensions. In some cases, a transducer array may determine a PWV at a maximum sampling rate, possibly employing ultrafast imaging. The sensors of the array may be disposed circumferentially about the distal portion of the flexible elongate member 170. In some embodiments, the sensors are not disposed circumferentially but rather along the axis of the flexible elongate member 170, and thereby do not detect the pressure/flow wave passing by measuring changes in vessel diameter, but by measuring changes in the distance of the sensors to the vessel wall.

In some embodiments, the use of sensors within a sensor array may allow for determination of a PWV without visualizing the propagation of wall distensions within the vessel. In this case, the PWV is determined according the following relation (where dQ is the change in flow within the vessel during a time interval, determined by integrating the flow profile (estimated by e.g. speckle tracking, vector flow, lateral oscillations, decorrelation) over the cross-section of the artery and dA is the change in cross-sectional area of the vessel during the time interval):

$$PWV = \frac{dQ}{dA} \qquad ( 3 )$$

In this case, the distance D1 between sensors 202, 204 should be small to enhance accuracy and enable estimation of the flow velocity profile. This flow velocity profile can be integrated over the vessel cross-section to determine the change in flow dQ. In some embodiments, a single array may be used. In some instances, at least one flow-sensing element is utilized to detect the flow from either within the vessel or from outside of the vessel. In some embodiments, dA may be determined by measuring the cross-sectional area of the vessel.

In some instances, the first and second sensors 202, 204 include components similar or identical to those found in IVUS products from Volcano Corporation, such as the Eagle Eye® Gold Catheter, the Visions® PV8.2F Catheter, the Visions® PV 018 Catheter, and/or the Revolution® 45 MHz Catheter, and/or IVUS products available from other manufacturers. Further, in some instances the intravascular system 100 and/or the intravascular device 110 includes components or features similar or identical to those disclosed in U.S. Pat. Nos. 4,917,097, 5,368,037, 5,453,575, 5,603,327, 5,779,644, 5,857,974, 5,876,344, 5,921,931, 5,938,615, 6,049,958, 6,080,109, 6,123,673, 6,165,128, 6,283,920, 6,309,339; 6,033,357, 6,457,365, 6,712,767, 6,725,081, 6,767,327, 6,776,763, 6,779,257, 6,780,157, 6,899,682, 6,962,567, 6,976,965, 7,097,620, 7,226,417, 7,641,480, 7,676,910, 7,711,413, and 7,736,317, each of which is hereby incorporated by reference in its entirety. The intravascular system 100 can incorporate the components associated with rotational and/or phased array IVUS apparatus, such as transducer(s), multiplexer(s), electrical connection(s), etc., for performing IVUS imaging, including grey-scale IVUS, forward-looking IVUS, rotational IVUS, phased array IVUS, solid state IVUS, and/or virtual histology.

In yet another example, the first and second sensors 202, 204 include an optical imaging element (e.g., a mirror, lens, prism, etc. and/or combinations thereof) in communication with coherent light source (e.g., a laser source) and a light detector such that optical coherence tomography (OCT) imaging can be used to determine the cross sectional area of the vessel. In some implementations, one or both of the sensors 202, 204 are optical acoustic transducers.

OCT systems operate in either the time domain or frequency (high definition) domain. In time-domain OCT, an interference spectrum is obtained by moving a scanning optic, such as a reference minor, longitudinally to change the reference path and match multiple optical paths due to reflections of the light within the sample. The signal giving the reflectivity is sampled over time, and light traveling at a specific distance creates interference in the detector. Moving the scanning mechanism laterally (or rotationally) across the sample produces reflectance distributions of the sample (i.e., an imaging data set) from which two-dimensional and three-dimensional images can be produced. In frequency domain OCT, a light source capable of emitting a range of optical frequencies passes through an interferometer, where the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample. Alternatively, in swept-source OCT, the interference spectrum is recorded by using a source with adjustable optical frequency, with the optical frequency of the source swept through a range of optical frequencies, and recording the interfered light intensity as a function of time during the sweep. Time- and frequency-domain systems can further vary based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, U.S. Pat. Nos. 5,321,501, 7,999,938; 7,995,210, 7,787,127, 7,783,337; 6,134,003; and 6,421,164, the content of each of which is incorporated by reference in their entireties.

Generally, the sensor 202 (and/or other similar sensors) can be used to obtain an imaging data from the vessel, from which the processing system 130 generates an intravascular image. The processing system 130 can determine one or more measurement values associated with the vessel, such as cross-sectional area, radius, diameter, wall thickness, and/or distance from the sensor to the vessel wall from the intravascular image.

Still referring to FIG. 1, the sensors 202, 204 may be disposed a distance D1 apart. In some embodiments, the distance D1 is fixed distance from 0.5 to 10 cm. In some embodiments, the distance D1 is within 0.5 to 2 cm. The distance D1 may be used in the calculation of Pulse Wave Velocity (PWV).

The sensors 202, 204 may be contained within the body of the intravascular device 110. The sensors 202, 204 may be disposed circumferentially around a distal portion of the intravascular device 110. In other embodiments, the sensors 202, 204 are disposed linearly along the intravascular device 110. The sensors 202, 204 may include one or more transducer elements. The sensor 202 and/or the sensor 204 may be movable along a length of the intravascular device 110 and/or fixed in a stationary position along the length of the intravascular device 110. The sensors 202, 204 may be part of a planar or otherwise suitably-shaped array of sensors of the intravascular device 110. In some embodiments, the outer diameter of the flexible elongate member 170 is equal to or larger than the outer diameter of the sensors 202, 204. In some embodiments, the outer diameter of the flexible elongate member 170 and sensors 202, 204 are equal to or less than about 1 mm, which may help to minimize the effect of the intravascular device 110 on the PWV determination within the vessel 80. In particular, since a renal artery generally has a diameter of approximately 5 mm, a 1 mm outer diameter of the intravascular device 110 may obstruct less than 4% of the vessel 80.

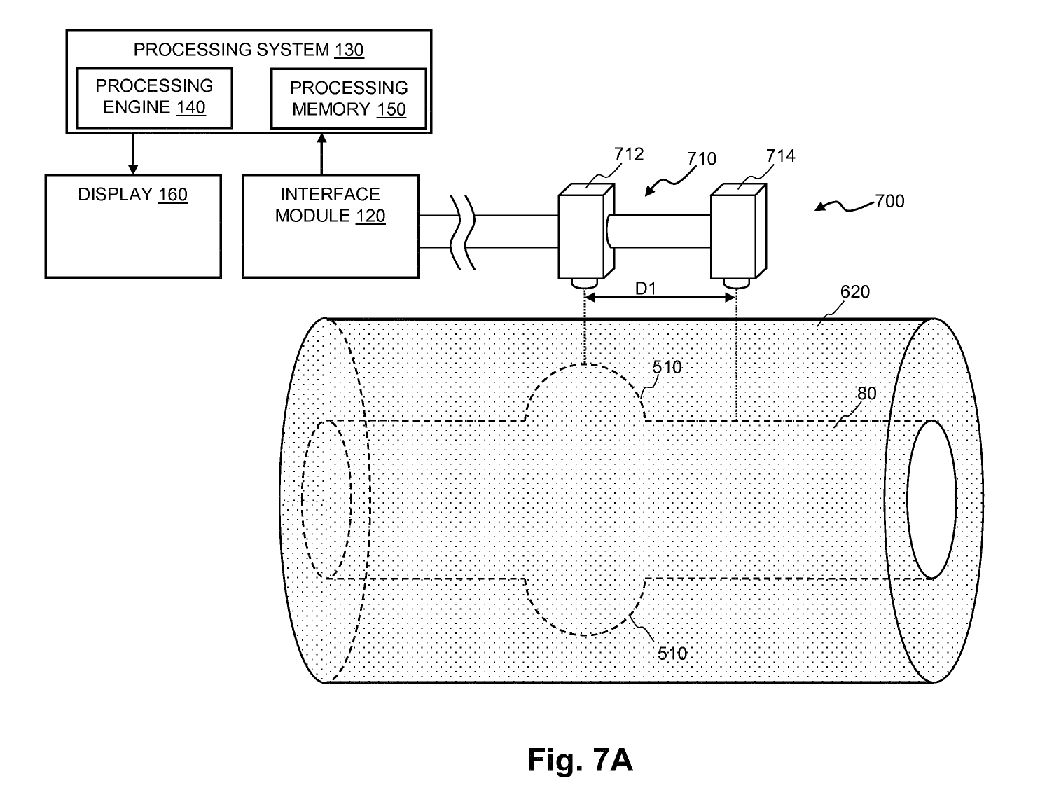

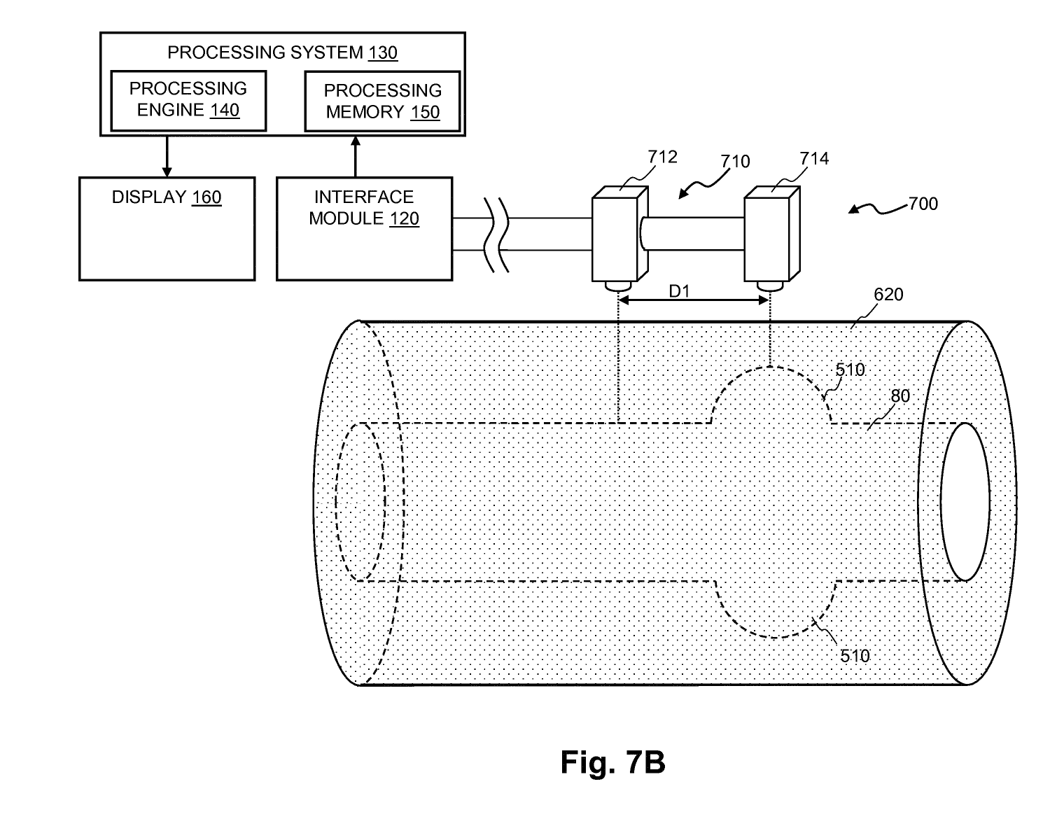

In some embodiments, one or both of the sensors 202, 204 may not be part of the intravascular device 110. For example, the sensor 204 may be coupled to a separate intravascular device or may be part of an external device. An example of sensors disposed externally is shown in relation to FIGS. 7A and 7B. For example, the sensor 204 may be coupled to one of a guide wire or a catheter, and the sensor 202 may be coupled to the other of the guide wire or the catheter. In some instances, a first intravascular device having one of the sensors 202, 204 may be a guide wire, and the second intravascular device having the other of the sensors 202, 204 may be a catheter. The first and second intravascular devices can be positioned side by side within the vessel 80 in some embodiments. In some embodiments, a guide wire can at least partially extend through and be positioned within a lumen of the catheter such that the catheter and guide wire are coaxial.

The processing system 130 may be in communication with the intravascular device 110. For example, the processing system 130 may communicate with the intravascular device 110, including the sensor 202 and/or the sensor 204, through an interface module 120. The processor 140 may send commands and receive responses from the intravascular device 110. In some implementations, the processor 140 controls the monitoring of one or more measurement values within the vessel 80 by the sensors 202, 204. The measurement value within the vessel 80 can include a vessel diameter, changes the vessel diameter, the distance between the sensors 202, 204 and the vessel walls, and/or changes in the distance between the sensors and the vessel walls. While some description herein may refer to vessel diameter, it is understood that any suitable measurement value within the vessel 80 is contemplated, including changes the vessel diameter, the distance between the sensors 202, 204 and the vessel walls, and/or changes in the distance between the sensors and the vessel walls. In particular, the processor 140 may be configured to trigger the activation of the sensors 202, 204 to measure, e.g., vessel diameter or other suitable measurement value at specific times. Data from the sensors 202, 204 may be received by a processor of the processing system 130. In other embodiments, the processor 140 is physically separated from the intravascular device 110 but in communication with the intravascular device 110 (e.g., via wireless communications). In some embodiments, the processor is configured to control the sensors 202, 204.

The processor 140 may include an integrated circuit with power, input, and output pins capable of performing logic functions such as commanding the sensors and receiving and processing data. The processor 140 may include any one or more of a microprocessor, a controller, a digital signal processor (DSP), an application specific integrated circuit (ASIC), a field-programmable gate array (FPGA), or equivalent discrete or integrated logic circuitry. In some examples, processor 140 may include multiple components, such as any combination of one or more microprocessors, one or more controllers, one or more DSPs, one or more ASICs, or one or more FPGAs, as well as other discrete or integrated logic circuitry. The functions attributed to processor 140 herein may be embodied as software, firmware, hardware or any combination thereof.

The processing system 130 may include one or more processors 140 or programmable processor units running programmable code instructions for implementing the pulse wave velocity determination methods described herein, among other functions. The processing system 130 may be integrated within a computer and/or other types of processor-based devices. For example, the processing system 130 may be part of a console, tablet, laptop, handheld device, or other controller used to generate control signals to control or direct the operation of the intravascular device 110. In some embodiments, a user may program or direct the operation of the intravascular device 110 and/or control aspects of the display 160. In some embodiments, the processing system 130 may be in direct communication with the intravascular device 110 (e.g., without an interface module 120), including via wired and/or wireless communication techniques.

Moreover, in some embodiments, the interface module 120 and processing system 130 are collocated and/or part of the same system, unit, chassis, or module. Together the interface module 120 and processing system 130 assemble, process, and render the sensor data for display as an image on a display 160. For example, in various embodiments, the interface module 120 and/or processing system 130 generate control signals to configure the sensors 202, 204, generate signals to activate the sensors 202, 204, perform calculations of sensor data, perform amplification, filtering, and/or aggregating of sensor data, and format the sensor data as an image for display. The allocation of these tasks and others may be distributed in various ways between the interface module 120 and processing system 130. In particular, the processing system 130 may use imaging data from the sensors 202, 204 to calculate a pulse wave velocity of the fluid (e.g., blood) inside the vessel 80.

The processing system 130 may be in communication with an electrocardiograph (ECG) console configured to obtain ECG data from electrodes positioned on the patient. ECG signals are representative of electrical activity of the heart and can be used to identify the patient's cardiac cycle and/or portions thereof. In some instances, the processing system 130 can utilize different formula to calculate PWV based on whether the vessel diameter data obtained by the intravascular device 110 is obtained over an entire cardiac cycle and/or a portion thereof. The ECG data can be used to identify the beginning and ending of the previous, current, and next cardiac cycle(s), the beginning and ending of systole, the beginning and ending of diastole, among other portions of the cardiac cycle. Generally, one or more identifiable feature of the ECG signal (including without limitation, the start of a P-wave, the peak of a P-wave, the end of a P-wave, a PR interval, a PR segment, the beginning of a QRS complex, the start of an R-wave, the peak of an R-wave, the end of an R-wave, the end of a QRS complex (J-point), an ST segment, the start of a T-wave, the peak of a T-wave, and the end of a T-wave) can utilized to select relevant portions of the cardiac cycle. The ECG console may include features similar or identical to those found in commercially available ECG elements such as the PageWriter cardiograph system available from Koninklijke Philips N.V.

Various peripheral devices may enable or improve input and output functionality of the processing system 130. Such peripheral devices may include, but are not necessarily limited to, standard input devices (such as a mouse, joystick, keyboard, etc.), standard output devices (such as a printer, speakers, a projector, graphical display screens, etc.), a CD-ROM drive, a flash drive, a network connection, and electrical connections between the processing system 130 and other components of the intravascular system 100. By way of non-limiting example, the processing system 130 may manipulate signals from the intravascular device 110 to generate an image on the display 160 representative of the acquired vessel diameter data, imaging data, PWV calculations, and/or combinations thereof. Such peripheral devices may also be used for downloading software containing processor instructions to enable general operation of the intravascular device 110 and/or the processing system 130, and for downloading software implemented programs to perform operations to control, for example, the operation of any auxiliary devices coupled to the intravascular device 110. In some embodiments, the processing system 130 may include a plurality of processing units employed in a wide range of centralized or remotely distributed data processing schemes.

The memory 150 may be a semiconductor memory such as, for example, read-only memory, a random access memory, a FRAM, or a NAND flash memory. The memory 150 may interface with the processor 140 such that the processor 140 may write to and read from the memory 150. For example, the processor 140 may be configured to receive data from the intravascular device 110 and/or the interface module 120 and write that data to the memory 150. In this manner, a series of data readings may be stored in the memory 150. The processor 140 may be capable of performing other basic memory functions, such as erasing or overwriting the memory 150, detecting when the memory 150 is full, and other common functions associated with managing semiconductor memory.

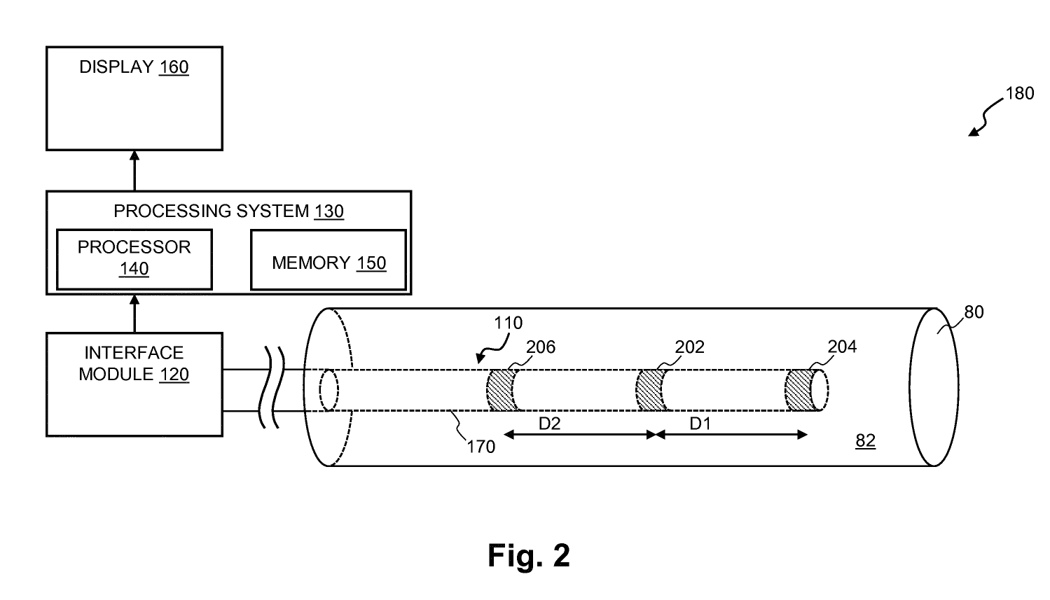

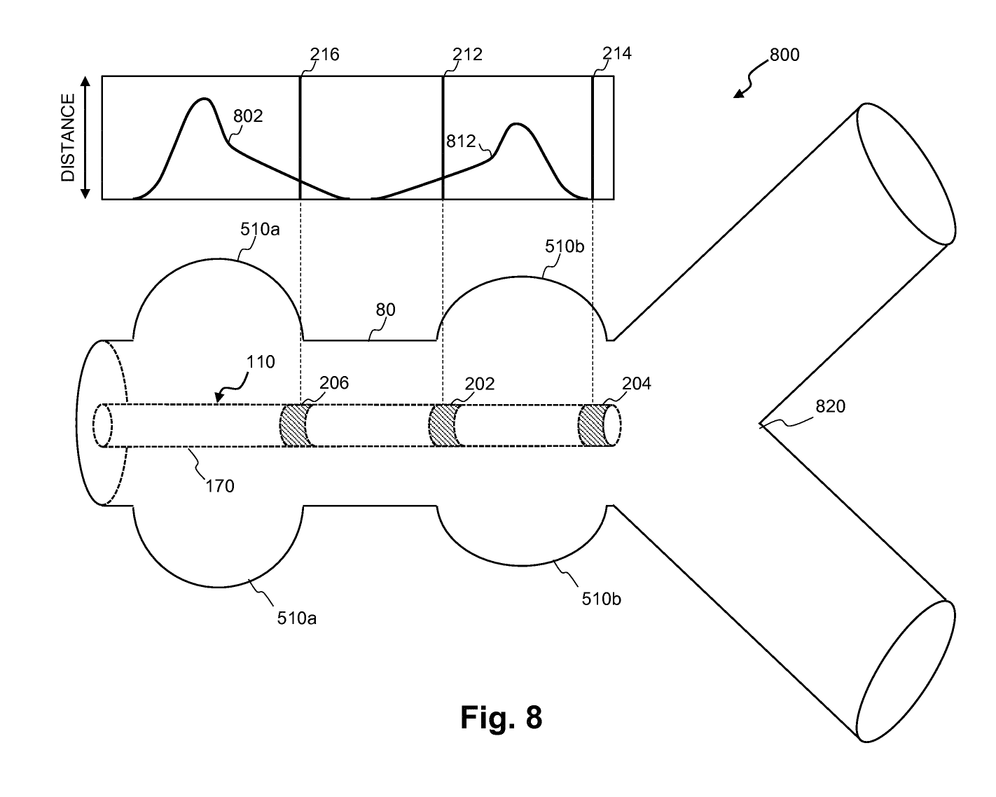

FIG. 2 is a diagrammatic schematic view of an exemplary intravascular system 180 according to some embodiments of the present disclosure. The intravascular system 180 may be similar to the intravascular system 100 of FIG. 1, with the addition of a third sensor 206. The intravascular systems as described herein may have four, five, six, or other numbers of sensors. The sensors may be placed in various orders and at different distances along the intravascular device 110. In some embodiments, the sensor 206 is disposed a distance D2 from the sensor 202. The sensors 202, 204, 206 may also be placed in other arrangements and orders than that shown in FIG. 2. The sensor 206 may have a similar functionality to the sensors 202, 204 and may be an ultrasound transducer configured to measure aspects of the vessel 80. In some embodiments, sensor 206 may be a pressure sensor. In some embodiments, the sensor 206 may be used to determine the direction of travel of various pulse waves travelling through the vessel 80. The determination of the direction of travel may enhance the accuracy of PWV determinations by allowing the elimination of backwards-travelling pulse waves and associated data. The methods associated with direction of travel determination are discussed in more detail in relation to FIG. 8.

FIG. 3 illustrates the intravascular device 110 of FIG. 1 disposed within the human renal anatomy. The human renal anatomy includes kidneys 10 that are supplied with oxygenated blood by right and left renal arteries 81, which branch off an abdominal aorta 90 at the renal ostia 92 to enter the hilum 95 of the kidney 10. The abdominal aorta 90 connects the renal arteries 81 to the heart (not shown). Deoxygenated blood flows from the kidneys 10 to the heart via renal veins 101 and an inferior vena cava 111. Specifically, the flexible elongate member 170 of the intravascular device 110 is shown extending through the abdominal aorta and into the left renal artery 81. In alternate embodiments, intravascular device 110 may be sized and configured to travel through the inferior renal vessels 115 as well. Specifically, the intravascular device 110 is shown extending through the abdominal aorta and into the left renal artery 81. In alternate embodiments, the intravascular device 110 may be sized and configured to travel through the inferior renal vessels 115 as well.

Left and right renal plexi or nerves 121 surround the left and right renal arteries 81, respectively. Anatomically, the renal nerve 121 forms one or more plexi within the adventitial tissue surrounding the renal artery 81. For the purpose of this disclosure, the renal nerve is defined as any individual nerve or plexus of nerves and ganglia that conducts a nerve signal to and/or from the kidney 10 and is anatomically located on the surface of the renal artery 81, parts of the abdominal aorta 90 where the renal artery 81 branches off the aorta 90, and/or on inferior branches of the renal artery 81. Nerve fibers contributing to the plexi arise from the celiac ganglion, the lowest splanchnic nerve, the corticorenal ganglion, and the aortic plexus. The renal nerves 121 extend in intimate association with the respective renal arteries into the substance of the respective kidneys 10. The nerves are distributed with branches of the renal artery to vessels of the kidney 10, the glomeruli, and the tubules. Each renal nerve 221 generally enters each respective kidney 10 in the area of the hilum 95 of the kidney, but may enter the kidney 10 in any location, including the location where the renal artery 81, or a branch of the renal artery 81, enters the kidney 10.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 121, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 121. Renal denervation may help alleviate the symptoms and sequelae of hypertension by blocking or suppressing the efferent and afferent sympathetic activity of the kidneys 10.

In some embodiments, the vessel 80 in FIG. 1 and FIG. 2 is a renal vessel consistent with the vessels 81 of FIG. 3 and the pulse wave velocity is determined in the renal artery. The processing system 130 may determine the pulse wave velocity (PWV) in the renal artery. The processing system 130 may determine a renal denervation therapy recommendation based on the pulse wave velocity in a renal artery. For example, patients that are more likely or less likely to benefit therapeutically from renal denervation may be selected based on the PWV. In that regard, based at least on the PWV of blood in the renal vessel, the processing system 130 can perform patient stratification for renal denervation.

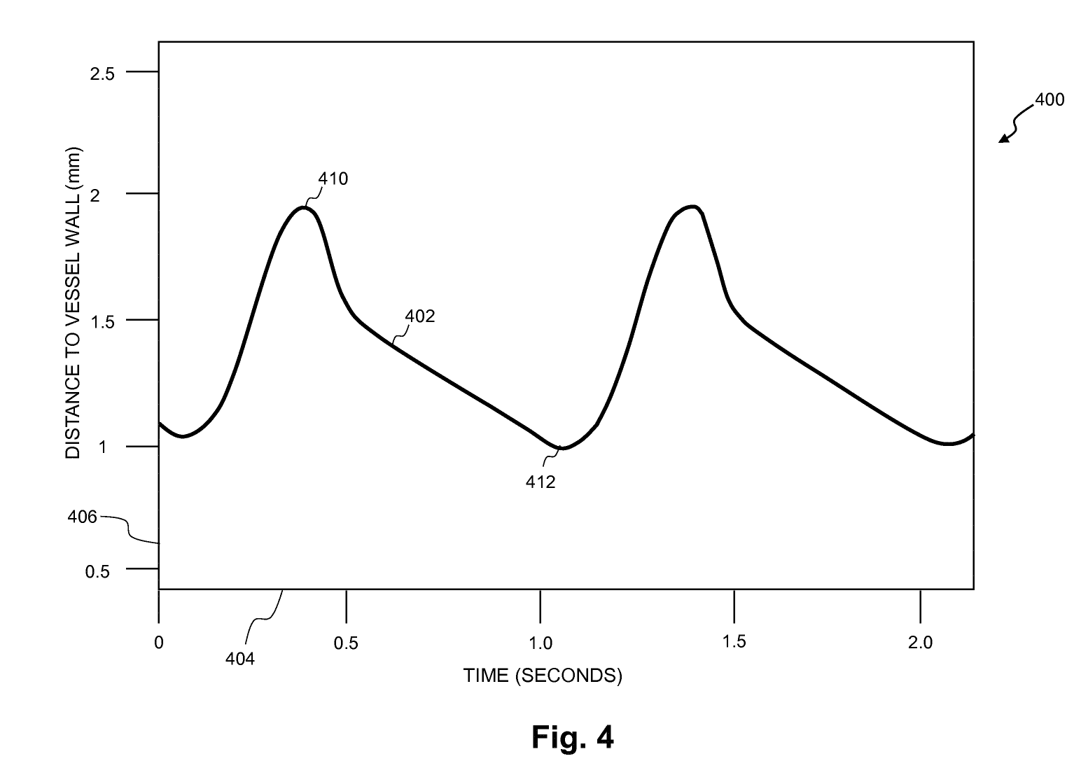

FIG. 4 is a graph 400 of measurements of the distance to the vessel wall associated with pulse waves travelling through a vessel. The graph 400 shows a curve 402 of a fluid, e.g., blood, travelling through a vessel. The horizontal axis 404 may represent time and the vertical axis 406 may represent the distance from the sensor (e.g., imaging element) to vessel wall from in arbitrary units. For example, the graph 400 shows two complete pulses, each one taking about 1 second (corresponding to a heart rate of approximately 60 beats per minute). As an example, the curve 402 of FIG. 4 may represent the pulse wave as a function of time at a specific point, e.g., the location of a sensor 202, 204, or 206 inside the vessel 80.

In some embodiments, pulse waves may be identified by certain aspects or characteristics of the distance curve 402 include including peaks 410, troughs 412, notches (e.g., dicrotic notches), minimum values, maximum values, changes in values, and/or recognizable pattern(s). Additionally, the pulse waves may be identified by a foot-to-foot analysis or by dedicated analysis of the pulse arrival time from the pulse waveform, as described in Solá et al, Physiological Measurement, vol. 30, pp. 603-615, 2009, which is incorporated by reference herein in its entirety. Alternatively, more generic methods for time delay estimation may be adopted for the assessment of the time delay between the pressure waves, such as cross-correlation analysis, phase transform methods, maximum likelihood estimators, adaptive least mean squares filters, average squared difference functions, or the multiple signal classification (MUSIC) algorithm. In some embodiments, sensors 202, 204, 206 may be configured to identify pulse waves by changes in the diameter of the vessel 80 or by changes in the distance between the sensors 202, 204, and 206 and the wall of the vessel 80. This sensor data may be used to determine the local PWV within a vessel 80. Optionally, the PWV value may then be used for stratification of patients with hypertension as eligible or ineligible for renal denervation.

The curve 402 may correspond to pressure waves within the vessel in some regard. That is, the pressure waves within the vessel may cause changes in the distance variation between the sensors 202, 204 and the vessel walls. The sensors 202, 204 need not measure pressure directly, but rather the imaging data obtained by the sensors 202, 204 may be used to determine the varying distances to the vessel walls caused by the pressure waves.

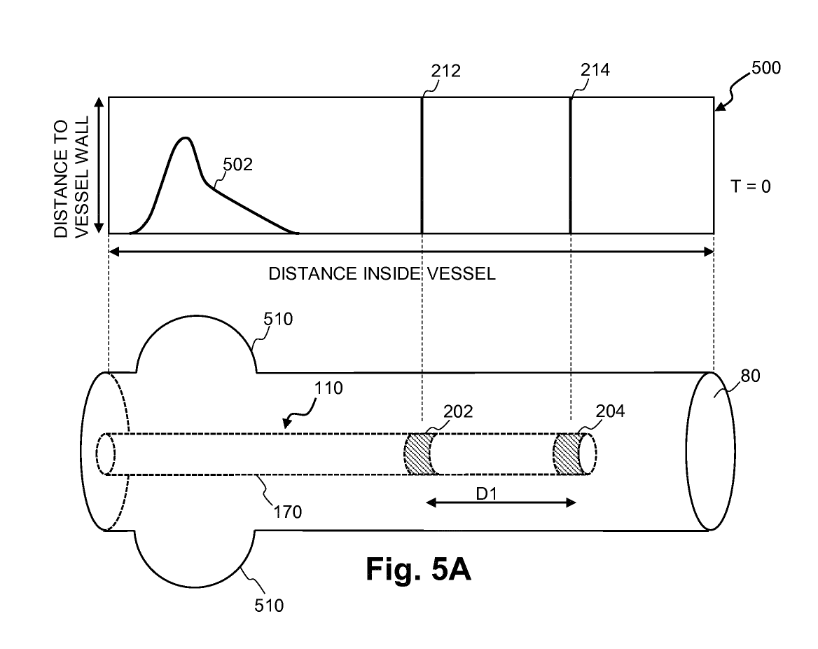

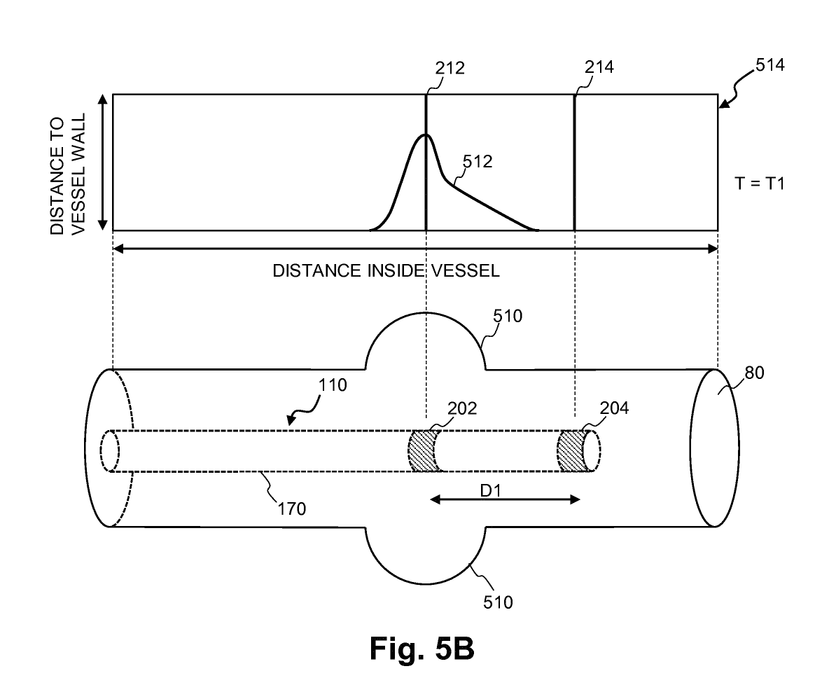

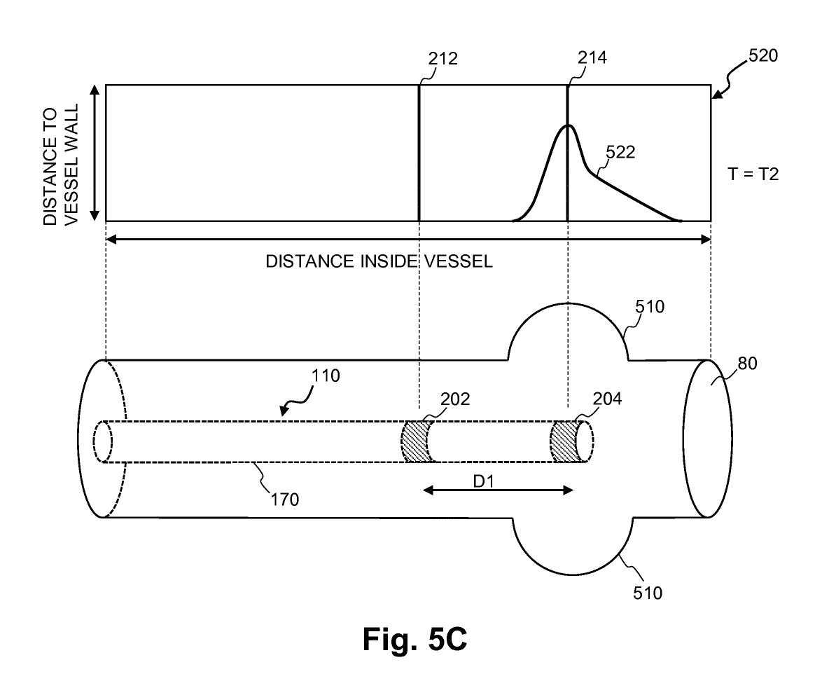

FIGS. 5A, 5B, and 5C show perspective views of an exemplary intravascular device 110 within a vessel 80 combined with a graph showing a distance of the imaging element to vessel wall curve within the vessel 80. The distance curve may be associated with a pulse wave travelling through the vessel 80 as discussed in relation to FIG. 4. In the example of FIG. 5A, the curve 502 of graph 500 shows a distance of an imaging element to the vessel wall when a pulse wave is travelling at the imaging element location at time T=0. The pressure by the pulse wave causes a moving distension 510 in the vessel wall. In particular, as the pulse wave travels through the vessel 80, the increased pressure causes a slight widening of the vessel 80. This distension 510 may be measured as an increase in vessel diameter by the first and second sensors 202, 204.

FIG. 5B shows the vessel at a later time T=T1. In this example, the pulse wave has moved to the right and the peak of the distance curve 512 on graph 514 is aligned at point 212 with the sensor 202. At this time T=T1, the sensor 202 will read a maximum increase in the diameter of the vessel 80 or a maximum distance between the sensor and the wall of the vessel which may be seen as distension 510, indicating the presence of the maximum pressure of the pulse wave at the point 212.

FIG. 5C shows the distance curve graph at a later time T=T2, where T2=T1+ΔT. The peak of the distance curve 522 on graph 520 is aligned with the sensor 204 at point 214. Thus, in the time period ΔT the pulse wave has traveled the distance D1 between the sensor 202 and the sensor 204. By dividing this distance D1 by the time period ΔT, the PWV may be calculated. That is,

$$PWV = \frac{{D}_{1}}{Δ t} ,$$

where D₁ is the distance between the sensors (e.g., imaging elements) 202 and 204, and Δt is the amount of time a pulse wave travelling between the first location of the sensor 202 and the second location of the sensor 204. Likewise, Δt can be described as a difference in the amount of time between the pulse wave reaching the sensor 202 and the pulse wave reaching the sensor 204. For example, the intravascular device 110 may include sensors 202, 204 disposed a distance D₁ of 2 cm apart. The sensor 202 may detect a distension 510 of the vessel 80 at time T=0. The sensor 204 may detect a distension 510 of the vessel 80 at time T=1 ms, making a time period ΔT of 1 ms. The PWV may be calculated by dividing D₁ by ΔT for a PWV of 20 m/s (0.02 m/0.001 s=20 m/s).

Due to the limited length of some vessels, such as the renal arteries 81, the sensors 202, 204 may be configured to collect imaging data at high frequencies to provide better accuracy. For example, to achieve 90% accuracy of a PWV while using the data from the above example in the calculation of PWV, the intravascular system 100 must be able to distinguish between 20 m/s and 18 m/s. If the speed is 18 m/s, the time period ΔT between the pulse wave arriving at the sensors 202, 204 is (0.02 m)/(18 m/s)=1.11 ms. Therefore, in order to distinguish these PWV values, the intravascular system 100 must be able to distinguish between a time period ΔT of 1 ms and 1.11 ms, and thus distinguish in the order of about 0.1 ms. The sampling frequency of an ultrasound transducer is limited by the time it takes for the ultrasound beam to propagate from the transducer to the vessel wall and back. Typically, the renal artery diameter is 5-6 mm. In case the transducer is placed against the wall, the ultrasound has to travel across twice the vessel diameter. Assuming a worst-case propagation distance of 15 mm, and given that the speed of sound in blood is about 1,570 m/s, it takes 0.0096 ms for the ultrasound to travel to the opposite vessel wall and back. This is about a factor of 10 lower than the 0.1 ms required for PWV determination, and sample rates up to 105 kHz can be reached. The intravascular system 100 may be able to achieve sampling frequencies in the order of 100 kHz (one measurement every 0.01 ms), allowing a delay of 0.1 ms to be detected. Preferably, the sampling frequency of the first and the second imaging element 202, 204 is 10 kHz or higher, more preferably, 20 kHz or higher, most preferably, 40 kHz or higher. In some embodiments, the sampling frequency of the intravascular system 100 is between 10 and 80 kHz, between 20 and 70 kHz, or between 40 and 60 kHz. Other ranges of sampling frequencies are also possible.

In some embodiments, the PWV may be determined by measuring movements in the vessel wall directly. The movement of the vessel wall may be used to locate pulse waves in the vessel. In some embodiments, vessel wall velocity may be measured with sensors using Doppler imaging. In particular, the movement of the vessel wall may be measured in two or more locations by the sensors 202, 204. By comparing the time delay associated with the wall velocity as measured by the various sensors, the PWV may be determined.

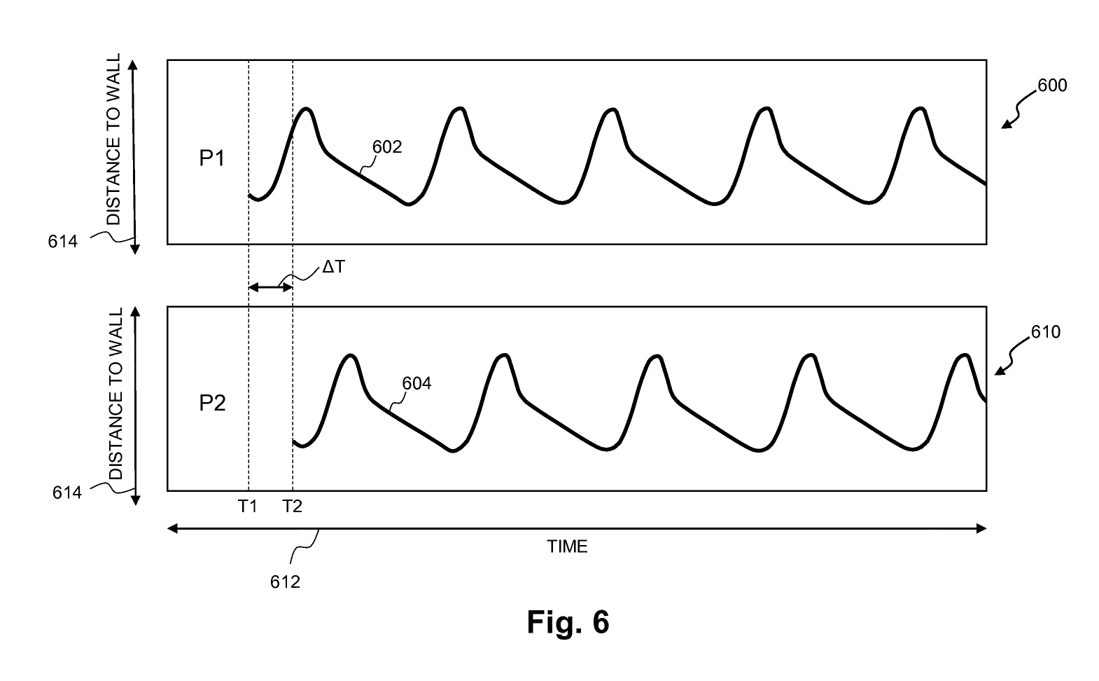

FIG. 6 shows two graphs associated with distance measurements of two sensors 202 and 204 measuring their distances to the vessel wall. Graph 600 shows the distance curve 602 of the distance between the imaging element 202 and the vessel wall when pressure waves of a fluid, e.g., blood, travels through the vessel at the location of the sensor 202, location P1 within the vessel. Graph 610 shows the distance curve 604 of the distance between the imaging element 204 and the vessel wall when the pulse waves travels through the vessel at the location of the sensor 204, location P2. In some embodiments, the distance curve 602, 604 may be determined by the intravascular system 100 through the collection and analysis of data from sensors such as the first and second sensors 202, 204. In some instances, the second location P2 is distal or downstream of the fluid flow from the first location. The horizontal axes 612 of the graphs 600 and 610 may represent time and the vertical axes 614 may represent the distance to vessel wall. As shown, the distance curve 602 of graph 600 starts at time T1 and the distance curve 604 of graph 610 starts at time T2, where ΔT=T2−T1 represents the time period it takes the pulse wave of the fluid to travel from the first location associated with graph 600 to the second location associated with graph 610. In this manner, the graphs 600 and 610 of FIG. 6 illustrate a pulse wave traveling along a vessel 80 where the pulse wave takes ΔT seconds to travel between first and second monitoring locations P1 and P2. This time period ΔT may be used to calculate the PWV of pulse waves in the vessel 80 as explained with reference to FIGS. 5A and 5B. In some examples, the curves 602, 604 are compared to determine ΔT and the comparison may be accomplished by a number of aspects, including as peaks, troughs, notches (e.g., dicrotic notches), minimum values, maximum values, changes in values, and/or recognizable pattern(s).

In some embodiments, the phase of the distance curves 602, 604 may be identified by comparing the measurements of the sensors 202, 204 at a given time. For example, the sensors 202 may collect imaging data showing a fluctuation of a vessel diameter or a fluctuation of the distance between sensor 202 and a wall of the vessel facing the sensor 202 over a period of time. In some embodiments, the activation of one or more of the sensors 202, 204 is delayed such that the distance curves 602, 604 measured by the sensors 202, 204 have the same phase. The delay required to match the phase of the distance curves 602, 604 is then used in the calculation of PWV. In some embodiments, the phase of the distance curves 602, 604 may be determined by actuating the first and second sensors 202, 204 simultaneously and comparing the vessel diameters from the sensors 202, 204. This method may include determining the delay by identifying when the difference between the vessel diameter measured by the sensors 202, 204 is zero. In some embodiments, the activation of the sensors 202, 204 is controlled by one or more of the interface module 120 or processing system 130 (as shown in FIGS. 1 and 2), which may include delaying the activation of sensors for certain time periods.

FIGS. 7A and 7B are diagrammatic schematic views of an exemplary measuring system 700 configured to measure PWV. The measuring system 700 may include an exterior device 710 that may be positioned outside a vessel 80, an interface module 120, a processing system 130 having at least one processor 140 and at least one memory 150, and a display 160, which may be similar to the components of FIG. 1. In some embodiments, the exterior device 710 may include two or more sensors 712, 714 configured to measure aspects of the vessel 80 from an external location. The sensors 712, 714 may be ultrasound transducers similar to the first, second, and third sensors 202, 204, 206. In some embodiments, the sensors 712, 714 measuring through the tissue 620 of a patient and determine the diameter of the vessel 80 or changes in the position of the vessel wall. In the example of FIG. 7A, a pulse wave is centered under the first sensor 712, which can be seen by the distension 510 of the vessel wall. In FIG. 7B, the pulse wave and associated distension 510 has traveled at distance D₁ and is centered under the second sensor 714. The distance D₁ between the sensors 712, 714 and the time difference in measurements of the distension 510 may be used to determine the PWV of the pulse wave.

FIG. 8 is a diagrammatic schematic view of an exemplary intravascular system 800 with an intravascular device 110 disposed within a vessel 80 combined with a graph 400 showing distance curves within the vessel 80. In some embodiments, pulse waves may be reflected within the vessel 80 for various reasons, including the presence of junctions or bifurcations 820 in the vasculature. This reflection may cause pulse waves to travel in different directions through the vessel 80 which may interfere with the measurement of local PWV values. However, in some embodiments, the intravascular device 110 may include three or more sensors 202, 204, 206 which may allow for the identification and exclusion of backward-travelling pulse waves by monitoring locations 212, 214, and 216, respectively. In particular, the third sensor 206 may be used to separate forward-travelling pulse waves (shown by curve 802 and distension 510a) from backward-travelling pulse waves (shown by curve 812 and distension 510b). In some embodiments, determining the directionality of the pulse waves may be accomplished by correlating ultrasound measurements from the three or more sensors 202, 204, 206 to identify the beginning and end of each pulse wave. The amplitude of the pulse waves and corresponding width of the distensions 510a, 510b may also be used in directionality determinations. For example, backward-travelling pulse waves such as that shown by distance curve 812 and distention 510b may have a smaller amplitude than forward-travelling pulse waves such as that shown by distance curve 802 and distension 510a. In some embodiments, the separation of forward- and backward-travelling pulse waves may improve the accuracy of PWV calculations.

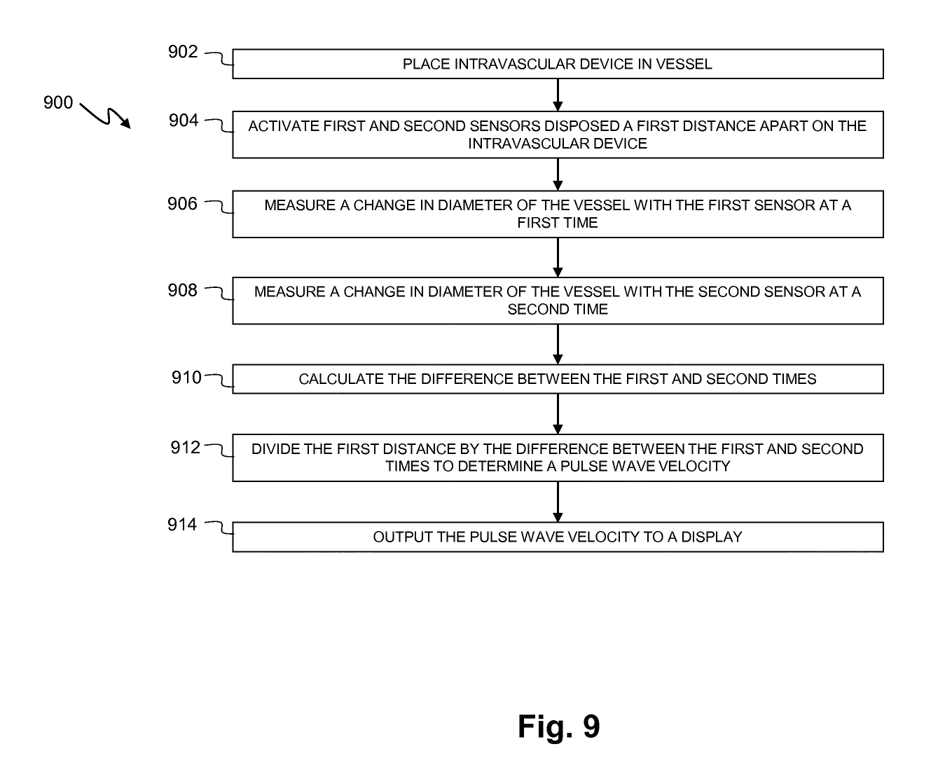

FIG. 9 is a flowchart illustrating a method 900 of calculating a pulse wave velocity (PWV). At step 902, the method 900 may include placing an intravascular device in a vessel. In some embodiments, the intravascular device is the intravascular device 110 shown in FIGS. 1, 2, 5A, 5B, 5C, and 8. The vessel may be a renal artery 81 as shown in FIG. 3.

At step 904, the method 900 may include activating first and second sensors disposed a first distance apart on the intravascular device. The first and second sensors may be disposed on a flexible elongate member. In other embodiments, the first and second sensors are disposed outside the body of the patients, such as in the example of FIGS. 7A and 7B. In some embodiments, intravascular imaging (e.g., intravascular ultrasound, rotational intravascular ultrasound, phased array intravascular ultrasound, or optical coherence tomography) is used to monitor a measurement value within the vessel, such as the vessel diameter or the distance between the sensors a vessel wall facing the sensors. In some embodiments, at least one of the first and second sensors is an ultrasound transducer. In other embodiments, at least one of the first and second sensors is an optical imaging element, such as a mirror, lens, prism, etc. The distance between the first and second sensors may be used in the calculation of the PWV. The first and second sensors may be disposed on a distal portion of a flexible, elongate device such as a catheter or guide wire. In some embodiments, an external probe (e.g., ultrasound imaging and/or Doppler flow) is used to monitor the vessel diameter.

At step 906, the method 900 may include measuring a change in the measurement value, such as the diameter of the vessel with the first sensor at a first time. Likewise, a change in the distance between the first sensor and the vessel wall can be measured. In some embodiments, the change in the diameter of the vessel or the change in the distance between the first sensor and the vessel wall may be a distension or bulge which may signal the presence of a pulse wave. The change can be a specific feature, for example, a peak of the diameter or a peal of the distance.

At step 908, the method 900 may include measuring a change in the measurement value, such as the diameter of the vessel with the second sensor at a second time. Likewise, a change in the distance between the second sensor and the vessel wall can be measured. This change in the diameter of the vessel or the change in the distance between the second sensor and the vessel wall may also be a distension or bulge which may signal the presence of a pulse wave. The change can be the same specific feature, for example, a peak of the diameter or a peal of the distance used in step 906 for the first sensor. In some embodiments, the direction of travel of the pulse wave may be determined, for example, by measuring the amplitude of distensions or by measuring change in the diameter of the vessel with additional sensors. Pulse waves that are travelling in a backwards direction (such as that shown in relation to FIG. 8) may be excluded from the calculation to improve the accuracy of the PWV determination.

At step 910, the method 900 may include calculating the difference between the first and second times. This difference may be similar to calculating the time period ΔT shown in relation to FIGS. 5C and 6. This calculation may be conducted by a controller in communication with the first and second sensors.

At step 912, the method 900 may include dividing the first distance by the difference between the first and second times to determine a PWV.

At step 914, the method 900 may optionally include outputting the PWV to a display. This display may be the display 160 shown in FIGS. 1 and 2. In some embodiments, the PWV may be used to evaluate the potential effect that renal denervation will have on a patient which may aid in selection of patients for whom renal denervation is likely beneficial.

In some embodiments, the method 900 optionally includes determining a therapy recommendation based on the PWV. In some instances, a clinician determines the therapy recommendation based on the computed PWV and/or other patient data. In some embodiments, the processing system evaluates the PWV and/or other patient data to determine the therapy recommendation. In such instances, the method 900 includes outputting a visual representation of the therapy recommendation. For example, the processing system can output display data associated with the graphical representation to a display device. The can be a textual indication, such as “Poor,” “Fair,” “Good,” and/or other suitable words may communicate the predicted benefit associated with therapy for the particular patient. In other instances, a numerical score, color coding, and/or other graphics representative of the therapy recommendation can be output to the display. The therapy can be renal denervation in some instances. The method 900 can additionally include classifying, based on the PWV, one or more patients into groups corresponding to respective degrees of predicted therapeutic benefit as a result of the renal denervation. The method 900 can also include the processing system outputting a graphical representation of the classifying step to the display device.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. An apparatus for pulse wave velocity (PWV) determination in a vessel, the apparatus comprising:
an intravascular device configured to be positioned within the vessel, the intravascular device including:
a flexible elongate member having a proximal portion and a distal portion;
a first imaging element coupled to the distal portion of the flexible elongate member; and
a second imaging element coupled to the distal portion of the flexible elongate member at a position spaced from the first imaging element by a first distance along a length of the flexible elongate member, wherein the first imaging element is configured to monitor a measurement value within the vessel at a first location, and wherein the second imaging element is configured to monitor the measurement value within the vessel (80) at a second location spaced from the first location; and
a processing system in communication with the intravascular device, the processing system configured to:
receive a first data associated with the monitoring of the measurement value of the vessel at the first location within the vessel by the first imaging element;
receive a second data associated with the monitoring of the measurement value of the vessel at the second location within the vessel by the second imaging element; and
determine a pulse wave velocity of fluid within the vessel based on the received first and second data,
wherein the vessel is a renal artery and the sampling frequency of the first and the second imaging element is 10 kHz or higher, more preferably, 20 kHz or higher, most preferably, 40 kHz or higher.

2. The apparatus of claim 1, wherein the measurement value comprises at least one of: a diameter of the vessel, a change in the diameter of the vessel, a distance to a wall of the vessel, or a change in the distance to the wall of the vessel.

3. The apparatus of claim 1, wherein the processing system is further configured to:
determine a renal denervation therapy recommendation based on the determined pulse wave velocity.

4. The apparatus of claim 1, wherein the processing system is further configured to:
classify a patient based on a predicted therapeutic benefit of renal denervation using the pulse wave velocity.

5. The apparatus of claim 1, wherein the pulse wave velocity is determined as
$$\frac{{D}_{1}}{Δ t} ,$$
where D₁ is the first distance and Δt is a difference in an amount of time between a pulse wave reaching the first location and the pulse wave reaching the second location.

6. The apparatus of claim 5, wherein an identifiable feature of the first and second data is utilized to determine the amount of time between the pulse wave reaching the first and second locations.

7. The apparatus of claim 6, wherein the identifiable feature is at least one of: a maximum diameter, a minimum diameter, or a slope.

8. The apparatus of claim 1, wherein the pulse wave velocity is determined as
$$\frac{dQ}{dA} ,$$
where dQ is a change in flow during a time interval and dA is a change in a cross-sectional area of the vessel during the time interval.

9. A method of determining pulse wave velocity (PWV) in a vessel, comprising:
monitoring a measurement value of the vessel at a first location of the vessel by a first imaging element;
monitoring a measurement value of the vessel at a second location of the vessel by a second imaging element, wherein the second location is spaced from the first location along a length of the vessel by a first distance;
receiving a first data associated with the monitoring of the measurement value of the vessel at the first location by the first imaging element;
receiving second data associated with the monitoring of the measurement value of the vessel at the second location by the second imaging element; and
determining a pulse wave velocity of fluid within the vessel based on the received first and second data,
wherein the vessel is a renal artery and the sampling frequency of the first and the second imaging element is 10 kHz or higher, more preferably, 20 kHz or higher, most preferably, 40 kHz or higher.

10. The method of claim 9, wherein the measurement value comprises at least one of: a diameter of the vessel, a change in the diameter of the vessel, a distance to a wall of the vessel, or a change in the distance to the wall of the vessel.

11. The method of claim 9, the method further comprising:
determining a renal denervation therapy recommendation based on the determined pulse wave velocity.

12. The method of claim 9, the method further comprising:
classifying a patient based on a predicted therapeutic benefit of renal denervation using the pulse wave velocity.

13. The method of claim 9, wherein the pulse wave velocity is determined as
$$\frac{{D}_{1}}{Δ t} ,$$
where D₁ is the first distance and Δt is an amount of time between a pulse wave reaching the first location and the pulse wave reaching the second location.

14. The method of claim 13, wherein an identifiable feature of the first and second data is utilized to determine the amount of time between the pulse wave reaching the first and second locations.

15. The method of claim 14, wherein the identifiable feature is at least one of: a maximum diameter, a minimum diameter, or a slope.

16. The method of claim 9, wherein the pulse wave velocity is determined as
$$\frac{dQ}{dA} ,$$
where dQ is a change in flow during a time interval and dA is a change in a cross-sectional area of the vessel during the time interval.

17. The method of claim 9, wherein the monitoring the measurement value of the vessel at the first location and the monitoring the measurement value of the vessel at the second location are performed using intravascular imaging.
